(19) United States
(12) Patent Application Publication
(10) Pub. No.: US 2010/0303896 A1
(43) Pub. Date: Dec. 2, 2010

(54) 5BETA, 14BETA-ANDROSTANE DERIVATIVES USEFUL FOR THE TREATMENT OF RESTENOSIS AFTER ANGIOPLASTIC OR ENDOARTHERECTOMY AND DISEASES DUE TO ORGAN FIBROSIS

(75) Inventors: Liberato Berrino, Castellamare Di Stabia (IT); Antonio Cascino, Napoli (IT); Marilena Cipollaro, Napoli (IT); Amalia Forte, Napoli (IT); Francesco Rossi, Napoli (IT); Giuseppe Bianchi, Milano (IT); Patrizia Ferrari, Varese (IT)

Correspondence Address: LUCAS & MERCANTI, LLP, 475 PARK AVENUE SOUTH, 15TH FLOOR, NEW YORK, NY, 10016, US

(73) Assignee: SIGMA-TAU INDUSTRIE FARMACEUTICHE RIUNITE S.P.A., Rome (IT)

(21) Appl. No.: 12/602,827

(22) PCT Filed: Jun. 4, 2008
(86) PCT No.: PCT/EP2008/056928

§ 371 Date: May 3, 2010

(30) Foreign Application Priority Data
Jun. 7, 2007 (EP) 07109779.4

(51) Int. Cl. A61K 9/127; A61K 31/58; A61K 9/00; A61P 1/16; A61P 9/00; A61P 17/02; A61P 19/04; A61P 13/12; A61P 35/00; A61P 11/00; A61P 1/18; A61F 2/82

(52) U.S. Cl. 424/450; 514/172; 514/176; 424/484; 623/1.43

(57) ABSTRACT

Compound of formula (I), wherein the symbol have the meaning reported in the text; for preparing a medicament for the prevention and/or treatment of obstructive vascular lesions following vascular surgery and for the prevention and/or treatment of diseases due to organ fibrosis.

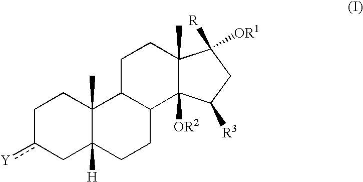

### FIELD OF THE INVENTION

The present invention relates to 17beta-(3-furyl) and (4-pyridazinyl)-5beta, 14beta-androstane derivatives, as useful agents for preparing a medicament for the prevention and treatment of restenosis after angioplastic or endoartherectomy, and diseases due to organ fibrosis.

More specifically the invention relates to compounds of formula (I):

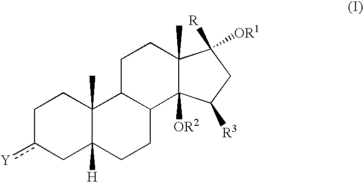

wherein:

the symbol [symbol] represents a single or a double bond;

Y is oxygen or guanidinoimino when [symbol] in position 3 is a double bond;

Y is hydroxy, O⁴ or SR⁴, when [symbol] in position 3 is a single bond and can have an alpha or beta configuration;

R is an unsubstituted or substituted 3-furyl or 4-pyridazinyl group;

R¹ is hydrogen; methyl; ethyl or n-propyl substituted by OH or NR⁵R⁶;

R² is hydrogen or together to R³ is a bond of an oxirane ring;

R³ is hydrogen or together to R² is a bond of an oxirane ring;

R⁴ is hydrogen; methyl; C2-C6 alkyl or C3-C6 alkenyl or C2-C6 acyl, these alkyl, alkenyl and acyl groups being unsubstituted or substituted by a quaternary ammonium group or one or more OR⁷, NR⁸R⁹, formyl, amidino, guanidinoimino or by NR⁸R⁹ and hydroxy;

R⁵, R⁶ are independently hydrogen; methyl; C2-C6 alkyl unsubstituted or substituted by one NR¹⁰R¹¹, or NR¹⁰R¹¹ and hydroxy, or R⁵ and R⁶ taken together with the nitrogen atom form an unsubstituted or substituted saturated or unsaturated penta- or hexa-monoheterocyclic ring, optionally containing another heteroatom chosen from oxygen or sulfur or nitrogen;

R⁷ is hydrogen, methyl or C2-C4 alkyl, this alkyl being unsubstituted or substituted by one or more NR¹⁰R¹¹ or by NR¹⁰R¹¹ and hydroxy;

R⁸, R⁹ are independently hydrogen; methyl; C2-C6 alkyl or C3-C6 alkenyl, these alkyl and alkenyl groups being unsubstituted or substituted by one or more NR¹⁰R¹¹, or NR¹⁰R¹¹ and hydroxy, or R⁸ and R⁹ taken together with the nitrogen atom form an unsubstituted or substituted saturated or unsaturated penta- or hexa-monoheterocyclic ring, optionally containing another heteroatom chosen from oxygen or sulfur or nitrogen, or R⁸ is hydrogen and R⁹ is amidino; or NR⁸R⁹ represents propargylamino,

R¹⁰, R¹¹ are independently hydrogen, C1-C6 alkyl, or R¹⁰ and R¹¹, taken together with the nitrogen atom form a saturated or unsaturated penta- or hexa-monoheterocyclic ring.

Also included in this invention are pharmaceutically acceptable salts of (I), which retain the biological activity of the base and are derived from such known pharmaceutically acceptable acids such as hydrochloric, sulfuric, phosphoric, malic, tartaric, maleic, citric, methanesulfonic or benzoic acid.

The alkyl and alkenyl groups may be branched or straight chain groups.

The C1-C6 alkyl group is preferably a C1-C4 alkyl group, e.g. methyl, ethyl, n-propyl, isopropyl, n-butyl, sec-butyl.

The C2-C6 alkyl group is preferably a C2-C4 alkyl group, e.g. ethyl, n-propyl, isopropyl, n-butyl, sec-butyl.

The C3-C6 alkenyl group is preferably a C3-C4 alkenyl group, e.g. 2-propenyl, 2-butenyl.

The C2-C6 acyl is preferably a C2-C4 acyl group, e.g. acetyl, propionyl, butyryl.

The quaternary ammonium group is preferably a trimethylammonium- or a N-methylpyrrolidinium- or a N-methylpiperidinium-group.

The OR⁷ group is preferably hydroxy, 2-aminoethoxy, 3-aminopropoxy, 2-dimethylaminoethoxy, 2-diethylaminoethoxy, 3-dimethylaminopropoxy, 3-amino-2-hydroxypropoxy, 2,3-diaminopropoxy, 2-(1-pyrrolidinyl)ethoxy, 3-(1-pyrrolidinyl)propoxy.

The NR⁵R⁶ group is preferably amino, methylamino, ethylamino, n-propylamino, dimethylamino, diethylamino, pyrrolidinyl, morpholino, piperazinyl, 1-imidazolyl, 2-aminoethylamino, 3-aminopropylamino.

The NR⁸R⁹ group is preferably amino, methylamino, ethylamino, n-propylamino, iso-propylamino, allylamino, propargylamino, dimethylamino, diethylamino, pyrrolidinyl, morpholino, piperazinyl, 1-imidazolyl, 1-guanidino, 2-aminoethylamino, 3-aminopropylamino, 2-(1-pyrrolidinyl)ethylamino, 3-(1-pyrrolidinyl)propylamino, 3-amino-2-hydroxypropylamino, 3-(1-pyrrolidinyl)2-hydroxypropylamino, 2,3-diaminopropylamino, (2-(1-pyrrolidinyl)ethyl)methylamino.

Preferred examples of specific compounds according to the present invention are:
17 beta-(3-Furyl)-5 beta-androstane-3 beta, 14 beta, 17 alpha-triol;
3 beta-(2-Hydroxyethoxy)-17 beta-(3-furyl)-5 beta-androstane-14 beta, 17 alpha-diol;
3 beta-(2-Aminoethoxy)-17 beta-(3-furyl)-5 beta-androstane-14 beta, 17 alpha-diol;
3 beta-(3-Aminopropoxy)-17 beta-(3-furyl)-5 beta-androstane-14 beta, 17 alpha-diol;
3 beta-(2-Methylaminoethoxy)-17 beta-(3-furyl)-5 beta-androstane-14 beta, 17 alpha-diol;
3 beta-(2-(1-Pyrrolidinyl)ethoxy)-17 beta-(3-furyl)-5 beta-androstane-14 beta, 17 alpha-diol;
3 beta-(2-(3-(1-Pyrrolidinyl)propoxy)ethoxy)-17 beta-(3-furyl)-5 beta-androstane-14 beta, 17 alpha-diol;
3 beta-(3-(1-Pyrrolidinyl)propoxy)-17 beta-(3-furyl)-5 beta-androstane-14 beta, 17 alpha-diol;
3 beta-(2-(1-Imidazolyl)ethoxy)-17 beta-(3-furyl)-5 beta-androstane-14 beta, 17 alpha-diol;
3 beta-(2-(2-Imidazolin-2-yl)ethoxy)-17 beta-(3-furyl)-5 beta-androstane-14 beta, 17 alpha-diol;
3 beta-(2-(2-Amidino)ethoxy)-17 beta-(3-furyl)-5 beta-androstane-14 beta, 17 alpha-diol;
3 beta-(2-(2-(1-Pyrrolidinyl)ethoxy)ethoxy)-17 beta-(3-furyl)-5 beta-androstane-14 beta, 17 alpha-diol;
beta-(2-Guanidinoethoxy)-17 beta-(3-furyl)-5 beta-androstane-14 beta, 17 alpha-diol;
beta-(3-Guanidinopropoxy)-17 beta-(3-furyl)-5 beta-androstane-14 beta, 17 alpha-diol;
3 beta-(3-Amino-2-hydroxypropoxy)-17 beta-(3-furyl)-5 beta-androstane-14 beta, 17 alpha-diol;
beta-(2,3-Diaminopropoxy)-17 beta-(3-furyl)5 beta-androstane-14 beta, 17 alpha-diol;
17 beta-(3-Furyl)-17 alpha-methoxy-5 beta-androstane-3 beta, 14 beta-diol;
17 beta-(3-Furyl)-17 alpha-(2-(1-pyrrolidinyl)ethoxy)-5 beta-androstane-3 beta, 14 beta-diol;
beta-(3-Furyl)-17 alpha-(3-aminopropoxy)-5 beta-androstane-3 beta, 14 beta-diol;
3 beta-(2-(1-Pyrrolidinyl)ethoxy)-17 beta-(3-furyl)-17 alpha-methoxy-5 beta-androstan-14 beta-ol;
3 beta, 17 alpha-Bis(2-(1-pyrrolidinyl)ethoxy)-17 beta-(3-furyl)-5 beta-androstan-14 beta-ol;
3 beta, 17 alpha-Bis(3-aminopropoxy)-17 beta-(3-furyl)-5 beta-androstan-14 beta-ol;
14 beta, 17 alpha-Dihydroxy-17 beta-(3-furyl)-5 beta-androstan-3-one;
3-Guanidinoimino-17 beta-(3-furyl)-5 beta-androstane-14 beta, 17 alpha-diol;
17 beta-(4-Pyridazinyl)-5 beta-androstane-3 beta, 14 beta, 17 alpha-triol;
3 beta-(2-Hydroxyethoxy)-17 beta-(4-pyridazinyl)5 beta-androstane-14 beta, 17 alpha-diol;
3 beta-(3-Aminopropoxy)-17 beta-(4-pyridazinyl)-5 beta-androstane-14 beta, 17 alpha-diol;
3 beta-(2-(1-Pyrrolidinyl)ethoxy)-17 beta-(4-pyridazinyl)-5 beta-androstane-14 beta, 17 alpha-diol;
3 beta-(3-(1-Pyrrolidinyl)propoxy)-17 beta-(4-pyridazinyl)-5 beta-androstane-14 beta, 17 alpha-diol;
17 beta-(4-Pridazinyl)-17 alpha-(3-aminopropoxy)-5 beta-androstane-3 beta, 14 beta-diol;
3 beta-(2-(1-Pyrrolidinyl)ethoxy)-17 beta-(4-pyridazinyl)-17 alpha-methoxy-5 beta-androstan-14 beta-ol;
3 beta-(2-(1-Pyrrolidinyl)ethoxy)-17 beta-(4-pyridazinyl)-17 alpha-(3-amino-propoxy)-5 beta-androstan-14 beta-ol;
14 beta, 17 alpha-Dihydroxy-17 beta-(4-pyridazinyl)-5 beta-androstan-3-one;
3-Guanidinoimino-17 beta-(4-pyridazinyl)-5 beta-androstane-14 beta, 17 alpha-diol;
14 beta, 15 beta-Epoxy-17 beta-(3-furyl)-5 beta-androstane-3 beta, 17 alpha-diol;
3 beta-(2-Hydroxyethoxy)-14 beta, 15 beta-epoxy-17 beta-(3-furyl)-5 beta-androstan-17 alpha-ol;
3 beta-(3-Aminopropoxy)-14 beta, 15 beta-epoxy-17 beta-(3-furyl)-5 beta-androstan-17 alpha-ol;
3 beta-(2-(1-Pyrrolidinyl)ethoxy)-14 beta, 15 beta-epoxy-17 beta-(3-furyl)-5 beta-androstan-17 alpha-ol;
3 beta-(3-(1-Pyrrolidinyl)propoxy)-14 beta, 15 beta-epoxy-17 beta-(3-furyl)-5 beta-androstan-17 alpha-ol;
3 beta-(2-(1-Pyrrolidinyl)ethoxy)-17 beta-(3-furyl)-17 alpha-methoxy-14 beta, 15 beta-epoxy-5 beta-androstane;
17 alpha-Hydroxy-17 beta-(3-furyl)-14 beta, 15 beta-epoxy-5 beta-androstan-3-one;
3-Guanidinoimino-17 beta-(3-furyl)-14 beta, 15 beta-epoxy-5 beta-androstan-17 alpha-ol;
14 beta, 15 beta-Epoxy-17 beta-(4-pyridazinyl)-5 beta-androstane-3 beta, 17 alpha-diol;

and the 3 alpha derivatives of the above identified 3 beta derivatives and also the corresponding 3 alpha and 3 beta thioderivatives where Y═S.

### BACKGROUND OF THE INVENTION

Restenosis is an obstructive lesion of the vessel that frequently occurs following surgery with mechanical angioplasty balloon (PTCA) or after endoartherectomy, performed for artery disease or organ transplantation.

This process has been considered a result of a succession of events: a) endothelial cells damage b) elastic recoil after the stretching of the artery c) neointimal hyperplasia due to proliferation and migration of vascular smooth muscle cells (SMC) d) remodelling and contraction of artery (Austin G E et al. J. Am. Coll. Cardiol. 6: 369-75, 1985).

In particular the neointimal hyperplasia leads to the re-obstruction of injured artery and is the result of platelet and leukocyte activation besides the proliferation of SMC. Pharmacological inhibition of restenosis often failed when drugs were administered by systemic delivery (Karthikeyan G and Bhargava B Curr. Opin. Cardiol. 19: 500-9. 2004).

Moreover, restenosis after angioplasty or endoartherectomy are due to damaged intima cells; angioplasty or endoartherectomy initiates a number of responses in the vessel wall including cellular migration, proliferation, and matrix accumulation, all of which contribute to neointima formation and restenosis (Malik N et al; Circulation 1998 Oct. 20; 98 (16): 1657-65). Inducing apoptosis may be beneficial also to reverse vascular disease, as pulmonary vascular disease (Cowan K. N. et al. Circ. Res. 1999 May 28; 84 (10): 1223-33).

A significant improvement in the prevention of restenosis has been realized with the implantation of a polymer stent at the site of surgery (Sigwart U et al. N. Engl. J. Med. 316: 701-716, 1987). In addition, the implantation of a stent offers the opportunity to vehicle drugs locally through a slow release (Laroia S T and Laroia A T Cardiol. Rev. 12: 37-43, 2004).

The present invention also relates to 17beta-(3-furyl) and (4-pyridazinyl)-5beta, 14beta-androstane derivatives as inhibitors of organ fibrosis, including kidney fibrosis, heart fibrosis, pancreas fibrosis, lung fibrosis, vascular vessel fibrosis, skin fibrosis, bone marrow fibrosis, liver fibrosis, and the like.

Acute or chronic lesion of the organs can be developed by a variety of factors including noxious materials such as virus, chemicals and undernourishment.

Ethanol is an extremely potent hepatotoxin and can lead to cirrhosis of the liver upon prolonged exposure. In fact 20% of chronic alcoholics will eventually experience cirrhosis. The process of cirrhosis of the liver involves a series of steps beginning with fatty infiltration which leads to necrosis or cell death, then fibrosis which in turn leads to cirrhosis.

Cirrhosis is not only the terminal stage of chronic liver diseases such as viral or alcoholic hepatitis, but also progresses highly frequently to hepatocellular carcinoma. Liver fibrosis is thought to be an outcome of excess deposition of extracellular matrices such as collagen during the repair of liver tissue when the balance is lost between hepatocyte necrosis triggered by an external factor, such as a virus and alcohol, or an internal factor involving autoimmune abnormality, and liver regeneration to maintain liver functions. At the cellular level, hepatocyte disorders and necrosis activate Kupffer's cells, endothelial cells and the like, so that TNF-.alpha., TGF-.beta., and PDGF are released from the activated Kupffer's cells and endothelial cells. It is considered that those factors then activate Hepatic stellate cells as the main factor of liver fibrosis, so that cellular growth and collagen synthesis are triggered.

Even in organs such as the lungs, kidneys, heart, pancreas, and skin, similarly to the liver, it is believed that fibroblasts existing in the individual organs and stromal cells specific to the individual organs (kidney mesangial cells, pancreatic stellate cells, etc.) lapse into abnormal growth and extracellular matrix synthesis due to the stimulation by various cytokines, leading to the occurrence of organ fibrosis.

17-(3-Furyl) and (4-pyridaziny)-5 beta, 14 beta-androstane derivative are known compound.

EP0583578B1 describes the beta-androstane derivatives claimed in the present application, a process for their preparation and their use for the treatment of cardiovascular disorders such as heart failure and hypertension.

EP0590271B1 describes 17-aryl and 17-heterocyclyl-5 alpha, 14 beta-androstane, androstene and androstadiene derivatives, a process for their preparation and their use for the treatment of cardiovascular disorders such as heart failure and hypertension.

EP 0590272B1 describes 17-Aryl and 17-heterocyclyl-5 beta, 14 beta-androstane derivatives and their use for the treatment of cardiovascular disorders such as heart failure and hypertension.

None of the publications above mentioned disclose the use of the 5beta, 14beta-androstane derivatives for the prevention and/or treatment of restenosis after angioplastic or endoartherectomy and organ fibrosis.

To date, no pharmaceutical agents effective as an organ fibrosis inhibitor have been sold on the market.

Therefore, the development of a pharmaceutical agent with a significant direct efficacy on organ fibrosis is needed.

It is therefore an object of the present invention the use of a compound of formula (I),

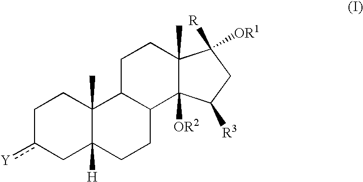

wherein the meaning of the substituents is mentioned above, for the prevention or treatment of restenosis.

It is a further object of the present invention the use of a compound of formula (I) for the prevention or treatment of diseases due to organ fibrosis.

Preferred examples of specific compounds of formula (I) are selected from the group consisting of:
17 beta-(3-Furyl)-5 beta-androstane-3 beta, 14 beta, 17 alpha-triol;
beta-(2-Hydroxyethoxy)-17 beta-(3-furyl)-5 beta androstane-14 beta, 17 alpha-diol;
3 beta-(2-Aminoethoxy)-17 beta-(3-furyl)-5 beta-androstane-14 beta, 17 alpha-diol;
3 beta-(3-Aminopropoxy)-17 beta-(3-furyl)-5 beta androstane-14 beta, 17 alpha-diol;
3 beta-(2-Methylaminoethoxy)-17 beta-(3-furyl)-5 beta-androstane-14 beta, 17 alpha-diol;
3 beta-(2-(1-Pyrrolidinyl)ethoxy)-17 beta-(3-furyl)-5 beta-androstane-14 beta, 17 alpha-diol;
3 beta-(2-(3-(1-Pyrrolidinyl)propoxy)ethoxy)-17 beta-(3-furyl)-5 beta-androstane-14 beta, 17 alpha-diol;
3 beta-(3-(1-Pyrrolidinyl)propoxy)-17 beta-(3-furyl)-5 beta-androstane-14 beta, 17 alpha-diol;
3 beta-(2-(1-Imidazolyl)ethoxy)-17 beta-(3-furyl)-5 beta-androstane-14 beta, 17 alpha-diol;
3 beta-(2-(2-Imidazolin-2-yl)ethoxy)-17 beta-(3-furyl)-5 beta-androstane-14 beta, 17 alpha-diol;
3 beta-(2-(2-Amidino)ethoxy)-17 beta-(3-furyl)-5 beta-androstane-14 beta, 17 alpha-diol;
3 beta-(2-(2-(1-Pyrrolidinyl)ethoxy)ethoxy)-17 beta-(3-furyl)-5 beta-androstane-14 beta, 17 alpha-diol;
3 beta-(2-Guanidinoethoxy)-17 beta-(3-furyl)5 beta-androstane-14 beta, 17 alpha-diol;
3 beta-(3-Guanidinopropoxy)-17 beta-(3-furyl)-5 beta-androstane-14 beta, 17 alpha-diol;
3 beta-(3-Amino-2-hydroxypropoxy)-17 beta-(3-furyl)-5 beta-androstane-14 beta, 17 alpha-diol;
3 beta-(2,3-Diaminopropoxy)-17 beta-(3-furyl)5 beta-androstane-14 beta, 17 alpha-diol;
17 beta-(3-Furyl)-17 alpha-methoxy-5 beta-androstane-3 beta, 14 beta-diol;
17 beta-(3-Furyl)-17 alpha-(2-(1-pyrrolidinyl)ethoxy)-5 beta-androstane-3 beta, 14 beta-diol;
17 beta-(3-Furyl)-17 alpha-(3-aminopropoxy)-5 beta-androstane-3 beta, 14 beta-diol;
3 beta-(2-(1-Pyrrolidinyl)ethoxy)-17 beta-(3-furyl)-17 alpha-methoxy-5 beta-androstan-14 beta-ol;
3 beta, 17 alpha-Bis(2-(1-pyrrolidinyl)ethoxy)-17 beta-(3-furyl)-5 beta-androstan-14 beta-ol;
3 beta, 17 alpha-Bis(3-aminopropoxy)-17 beta-(3-furyl)-5 beta-androstan-14 beta-ol;
14 beta, 17 alpha-Dihydroxy-17 beta-(3-furyl)-5 beta-androstan-3-one;
3-Guanidinoimino-17 beta-(3-furyl)-5 beta-androstane-14 beta, 17 alpha-diol;
17 beta-(4-Pyridazinyl)-5 beta-androstane-3 beta, 14 beta, 17 alpha-triol;
3 beta-(2-Hydroxyethoxy)-17 beta-(4-pyridazinyl)5 beta-androstane-14 beta, 17 alpha-diol;
3 beta-(3-Aminopropoxy)-17 beta-(4-pyridazinyl)-5 beta-androstane-14 beta, 17 alpha-diol;
3 beta-(2-(1-Pyrrolidinyl)ethoxy)-17 beta-(4-pyridazinyl)-5 beta-androstane-14 beta, 17 alpha-diol;
3 beta-(3-(1-Pyrrolidinyl)propoxy)-17 beta-(4-pyridazinyl)-5 beta-androstane-14 beta, 17 alpha-diol;
17 beta-(4-Pridazinyl)-17 alpha-(3-aminopropoxy)-5 beta-androstane-3 beta, 14 beta-diol;
3 beta-(2-(1-Pyrrolidinyl)ethoxy)-17 beta-(4-pyridazinyl)-17 alpha-methoxy-5 beta-androstan-14 beta-ol;
3 beta-(2-(1-Pyrrolidinyl)ethoxy)-17 beta-(4-pyridazinyl)-17 alpha-(3-amino-propoxy)-5 beta-androstan-14 beta-ol;
14 beta, 17 alpha-Dihydroxy-17 beta-(4-pyridazinyl)-5 beta-androstan-3-one,
3-Guanidinoimino-17 beta-(4-pyridazinyl)-5 beta-androstane-14 beta, 17 alpha-diol;
14 beta, 15 beta-Epoxy-17 beta-(3-furyl)-5 beta-androstane-3 beta, 17 alpha-diol;
3 beta-(2-Hydroxyethoxy)-14 beta, 15 beta-epoxy-17 beta-(3-furyl)-5 beta-androstan-17 alpha-ol;
3 beta-(3-Aminopropoxy)-14 beta, 15 beta-epoxy-17 beta-(3-furyl)-5 beta-androstan-17 alpha-ol;
3 beta-(2-(1-Pyrrolidinyl)ethoxy)-14 beta, 15 beta-epoxy-17 beta-(3-furyl)-5 beta-androstan-17 alpha-ol;
3 beta-(3-(1-Pyrrolidinyl)propoxy)-14 beta, 15 beta-epoxy-17 beta-(3-furyl)-5 beta-androstan-17 alpha-01;
3 beta-(2-(1-Pyrrolidinyl)ethoxy)-17 beta-(3-furyl)-17 alpha-methoxy-14 beta, 15 beta-epoxy-5 beta-androstane;
17 alpha-Hydroxy-17 beta-(3-furyl)-14 beta, 15 beta-epoxy-5 beta-androstan-3-one;
3-Guanidinoimino-17 beta-(3-furyl)-14 beta, 15 beta-epoxy-5 beta-androstan-17 alpha-ol;
14 beta, 15 beta-Epoxy-17 beta-(4-pyridazinyl)-5 beta-androstane-3 beta, 17 alpha-diol; or the 3 alpha derivatives of the above identified 3 beta derivatives and also the corresponding 3 alpha and 3 beta thioderivatives where Y═S.

The most preferred example of specific compound according to the present invention is 17 beta-(3-Furyl)-5 beta-androstane-3beta, 14 beta, 17 alpha-triol, in the following mentioned as “rostafuroxin”.

It is a further object of the present invention the use of a compound of formula (I) for the preparation of a medicament for the treatment of obstructive vascular lesions following vascular surgery, such as for example after angioplasty, percutaneous transluminal coronary angioplasty (PTCA), bypass grafting, endartherectomy or stent implantation. Preferably the pathological conditions to be treated according to the invention are restenosis after angioplastic or after endoartherectomy;

It is a further object of the present invention the use of a compound of formula (I) for the preparation of a medicament for the prevention or treatment of diseases due to organ fibrosis such as: kidney fibrosis due to diabetic nephropathy, glomerulonephritis, or nephrosclerosis; heart fibrosis due to chronic coronary insufficiency or aging; pancreas fibrosis due to pancreatic diseases; lung fibrosis due to lung diseases; vascular vessel fibrosis due to vascular degenerative diseases such as arteriosclerosis or restenosis after vascular disobliteration following Coronary by-pass surgery and percutaneous transluminal coronary angioplasty (PTCA) or shunt insertion; skin fibrosis; bone marrow fibrosis; liver fibrosis due to virus and alcoholic chronic hepatitis, non-alcoholic steatohepatitis (NASH), cirrhosis and liver cancer; and other organs fibrosis due to diseases such as pachyderma, keloid and systemic sclerosis.

It is a further object of the present invention a method of treating a mammal suffering from obstructive vascular lesions following vascular surgery, comprising administering a therapeutically effective amount of a compound of formula (I). The term “therapeutically effective amount” as used herein refers to an amount of a therapeutic agent needed to treat, ameliorate a targeted disease or condition, or to exhibit a detectable therapeutic effect.

The precise effective amount for a human subject will depend upon the severity of the disease state, general health of the subject, age, weight, and gender of the subject, diet, time and frequency of administration, drug combination (s), reaction sensitivities, and tolerance/response to therapy. This amount can be determined by routine experimentation and is within the judgement of the clinician. Generally, an effective dose per day will be from 0.05 mg to 20 mg, preferably 0.5 mg to 15 mg, most preferably 5 mg to 10 mg of a compound of formula (1), preferred is rostafuroxin.

Dosage treatment may be a single dose schedule or a multiple dose schedule, according to the physician judgement.

Compositions may be administered individually to a patient or may be administered in combination with other agents, drugs or hormones.

The medicament may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity.

Suitable carriers may be large, slowly metabolised macromolecules such as proteins, polysaccharides, polylactic acids, polyglycolic acids, polymeric amino acids, amino acid copolymers and inactive virus particles.

A thorough discussion of pharmaceutically acceptable carriers is available in Remington's Pharmaceutical Sciences (Mack Pub. Co., N. J. 1991).

Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The medicament of this invention may be administered by any number of routes including, but not limited to, oral, intravenous, intramuscular, intra-arterial, intramedullary, intrathecal, intraventricular, transdermal or transcutaneous applications, subcutaneous, intraperitoneal, intranasal, enteral, topical, sublingual, rectal means or locally on the diseased tissue after surgical operation. The compound of the invention may also be applied (coated) on the stent even incorporated into a controlled-release matrix.

### DISCUSSION OF THE DRAWINGS

The drawings show the effect of rostafuroxin (1) in the prevention of restenosis after endoartherectomy in MHS and MNS rats (hematoxilin-orcein staining, 10× magnification); and (2) in the prevention of liver fibrosis in rats (Trichrome stain, 200× magnification).

FIG. 1(A) cross-section of MHS carotid not injured of a rat not treated.

FIG. 1 (B) cross-section of MHS carotid injured, harvested 30 days after surgery, of a rat treated orally with Methocel 0.5%.

FIG. 1 (C) cross-section of MHS carotid injured, harvested 30 days after surgery, of a rat treated orally with Rostafuroxin 100 μg/kg.

FIG. 2(A) cross-section of MNS carotid not injured of a rat not treated.

FIG. 2 (B) cross-section of MNS carotid injured, harvested 30 days after surgery, of a rat treated orally with Methocel 0.5%.

FIG. 2 (C) cross-section of MNS carotid injured, harvested 30 days after surgery, of a rat treated orally with Rostafuroxin 100 μg/kg.

FIG. 3 shows normal staining patterns of the liver in control rat (Ctr. group).

FIG. 4 shows that CCl4 liver injury induces remarkable hepatocellular damage which in turn stimulates the onset of a complex wound healing process, leading to hepatic fibrosis (Ctr. LF group).

FIG. 5 shows that in Rostafuroxin treated animals (RLF group), less extended tissue damage is associated to a minor degree of liver fibrosis.

FIG. 6 shows normal staining pattern of the liver in control rat (Ctr. group): score 0.

FIG. 7 shows the histological appearance of liver from two CCl4 treated rats (Crt. LF group) with fibrotic lesions of moderate degree: score 2.

FIG. 8 shows the histological appearance of liver from two CCl4 rats treated with Rostafuroxin at 1 mg/kg os (RLF group) with fibrotic lesions of mild degree: score 1.

The following non-limiting examples further illustrate the invention.

### EXAMPLE 1

To test the activity of the compound of the invention for the prevention of restenosis after endoartherectomy, spontaneous hypertensive MHS rats with genetic arterial hypertension (Bianchi G., Barber B. R., Torielli L., Ferrari P. The Milan hypertensive strain of rats. Genetic Model of Hypertension. 1994; 22: 457-460) were used. MHS rats are available at Prassis Research Institute, Sigma-tau, Italy.

Two groups of 25-day-old MHS rats were orally treated by gavage with vehicle (Methocel 05.%) or Rostafuroxin (100 g/kg) for 6 weeks. After this period rats were subjected to the vascular surgical injury according to the protocol listed below. After surgical injury, rats were maintained under treatment either with vehicle or Rostafuroxin for further 30 days and then sacrificed for carotid artery sampling. Some post-surgery mortality was observed in the two rat strains. The final number of rats used for the histological carotid analysis was; MHS vehicle=7, MHS Rostafuroxin=9.

Vascular Injury

The carotid surgical injury model used on MHS and MNS rat is described in J. Cell. Physiol. 2001; 186:307-313.

Briefly, a plastic Scanlon clamp for coronary artery bypass grafting was placed for 10 seconds on the carotid artery in order to cause a crushing lesion to vessel. At the same point where the clamp was applied, a 0.5 mm longitudinal incision was performed on the full thickness of the carotid artery. The incision did not cross to the other side of vessel. Hemostasis was obtained with a single adventitial 8.0-gauge polypropylene stitch. Once bleeding stopped, the carotid artery was carefully examined and blood pulsation was checked distally to the incision.

Histological Analysis

Carotid arteries were taken 30 days after injury. Rats were anaesthetized and carotids were dissected free from the surrounding tissues. Through thoracotomy, the left ventricle of the beating heart was cannulated with a blunt syringe. The syringe was held in place by a ligature in the ascending aorta. The vessels were perfused at physiological pressure first with saline until the effluent was clear, and then perfusion fixed with 4% buffered (pH 7) formaldehyde. An incision in the right atrium served as outflow tract. Before switching to formaldehyde, the descending thoracic aorta was clamped. Tissue samples were taken 20 min after perfusion. Samples were further fixed in 4% formaldehyde o.n., dehydrated and finally embedded in paraffin. Cross-sections (5 μm) were stained with hematoxylin-orcein for elastic fiber analysis.

For each injured carotid, at least 60 serial cross-sections were observed under a light microscope at 20× magnification; image screening and photography were performed using Leica IM 1000 System (Leica, Wetzlar, Germany). The sections of injured carotids showing maximal remodelling and proliferative phenomena were identified and further analyzed.

Lumen and medial areas were measured using the Leica IM 1000 software (Leica, Heerbrugg, Switzerland). The former was defined as the area enclosed by internal elastic lamina, while the latter was defined as the area enclosed between the external and internal elastic laminae. In order to reduce individual rat variability, the lumen and the medial areas of each treated carotid were normalized with respect to the contralateral uninjured carotid. For each contralateral uninjured carotid, at least 10 sections were analyzed and the average media and lumen areas were calculated.

Measurements were performed by two independent observers.

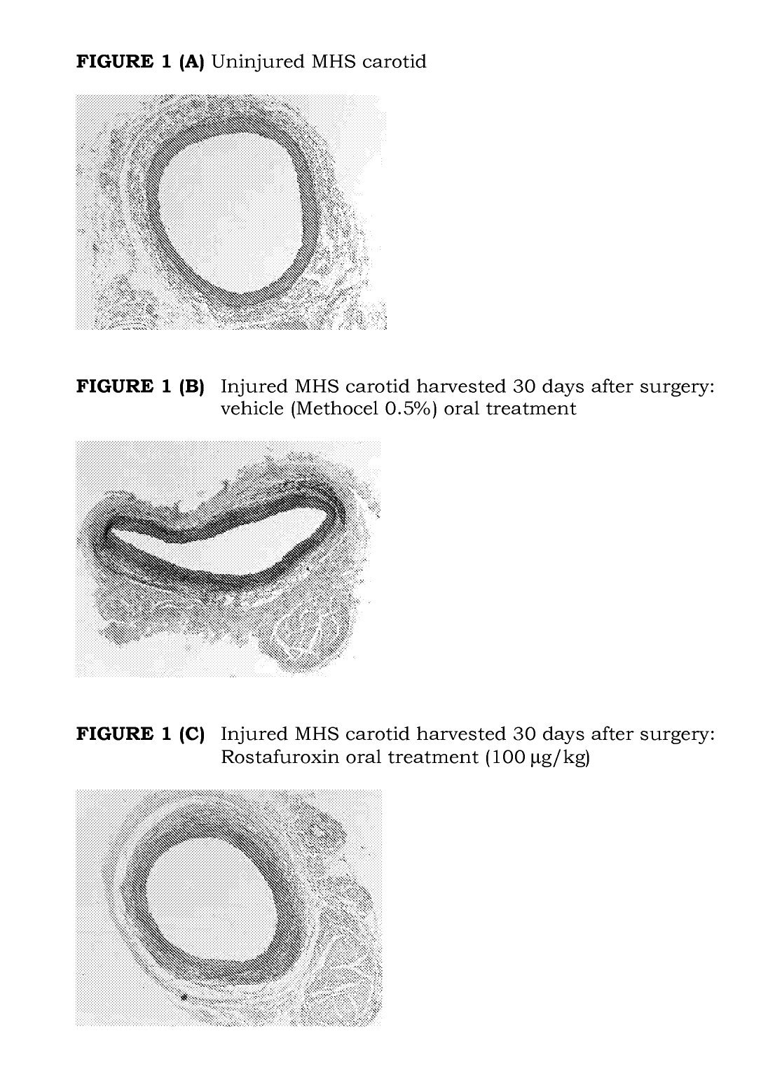

The results obtained are reported in the following Table 1 and FIG. 1(A-C)

TABLE 1
Morphometric measurements of the carotid lumen
area of spontaneous hypertensive MHS rats.

Injured/Uninjured carotid ratio

| | | Rostafuroxin |
|---|---|---|
| Animal n° | Methocel 0.5% | 100 μg/kg |
| 1 | 0.65 | 0.90 |
| 2 | 0.48 | 0.89 |
| 3 | 0.41 | 0.86 |
| 4 | 0.35 | 0.83 |
| 5 | 0.31 | 0.81 |
| 6 | 0.23 | 0.66 |
| 7 | 0.22 | 0.54 |
| 8 | — | 0.36 |
| 9 | — | 0.25 |
| mean | 0.379 | 0.680 |
| ±SD | 0.057 | 0.081 |
| Student't test | | p < 0.0094 |

The results reported in Table 1/FIG. 1 (A-C) show that the compound of the invention reduced in statistically significant manner (−45%) the arterial restenosis in MHS rats with arterial hypertension.

### EXAMPLE 2

To results reported in Example 1 were confirmed using MNS rats without arterial hypertension. The method used was the same as described in Example 1.

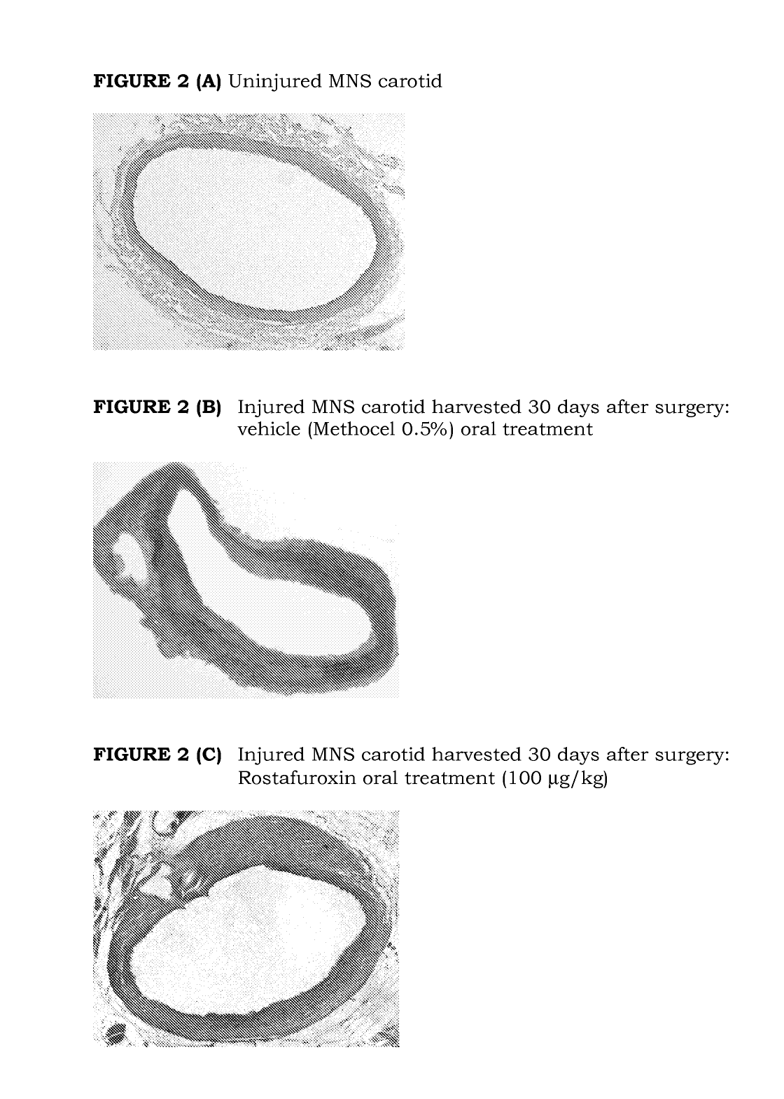

The results obtained are reported in the following Table 2 and FIG. 2(A-C).

TABLE 2
Morphometric measurements of the carotid lumen
area of MNS rats without arterial hypertension.

Injured/Uninjured carotid ratio

| | | Rostafuroxin |
|---|---|---|
| Animal n° | Methocel 0.5% | 100 μg/kg |
| 1 | 0.94 | 1.00 |
| 2 | 0.80 | 0.95 |
| 3 | 0.65 | 0.93 |
| 4 | 0.60 | 0.83 |
| 5 | 0.58 | 0.80 |
| 6 | 0.56 | 0.76 |
| 7 | 0.53 | 0.75 |
| 8 | 0.51 | 0.68 |
| 9 | 0.49 | 0.59 |
| 10 | 0.47 | 0.36 |
| 11 | 0.45 | — |
| 12 | 0.38 | — |
| 13 | 0.36 | — |
| 14 | 0.24 | — |
| Mean | 0.540 | 0.765 |
| ±SD | 0.047 | 0.060 |
| Student't test | | p < 0.0084 |

The results reported in Table 2, which are statistically significant, confirmed the results obtained in Example 1.

### EXAMPLE 3

To test the activity of the compound of the invention for the prevention and treatment of organ fibrosis, spontaneous hypertensive MHS rats with genetic arterial hypertension (Bianchi G., Barber B. R., Torielli L., Ferrari P. The Milan hypertensive strain of rats. Genetic Model of Hypertension. 1994; 22: 457-460) were used. MHS rats are available at Prassis Research Institute, Sigma-tau, Italy.

Liver fibrosis was induced by CCl₄ oral administration as described in J. Hepatol. 1999; 30: 621-631, with minor modifications: 0.375 ml/kg of CCl₄ dissolved in olive oil was administered three times per week for three weeks by oral gavage to rats starting from the 7^{th} week after weaning. Control rats received only the vehicle (olive oil). Rostafuroxin treatment started 7 weeks before the induction of liver fibrosis by CCl₄ administration (pretreatment) and was continued during the CCl₄ treatment for three weeks. Rostafuroxin was orally administered at 1 mg/kg/day, suspended in Methocel (0.5%). A total of twenty four MHS rats of 25 days of age (weaning) were subdivided in three groups of 8 rats each: the first group, considered as negative control for liver fibrosis induction (Ctr.), was orally treated with the Rostafuroxin vehicle (Methocel 0.5%) along the entire period (10 weeks) and received the olive oil as vehicle of CCl₄ at the 7^{th} week after weaning for three weeks; the second group, considered as positive control for liver fibrosis (Ctr. LF), was orally treated with the Rostafuroxin vehicle (Methocel 0.5%) along the entire period (10 weeks) and received CCl₄ at the 7^{th} week after weaning for three weeks; the third group, Rostafuroxin treated with liver fibrosis (RLF), received Rostafuroxin at 1 mg/kg along the entire period (10 weeks) and received CCl₄ at the 7^{th} week after weaning for three weeks. At the end of the treatment (10^{th} week), rats were weighed and anaesthetized with pentobarbital for blood sampling and organ removal. The blood was let clotted and serum separated after centrifugation and saved for measurement of serum albuminemia and transaminases (ALT and AST) on a Pentra 400 (ABX) Automatic Hematology Analyzer. Spleen and liver were removed and weighed. Samples of the liver median lobe were immediately frozen in liquid nitrogen and kept at −80° for further biochemical measurement of the 4-Hydroxiproline liver content, taken as an index of collagen deposition (Biochem. J. 1961; 80(1): 148-154). The rest of the liver was preserved in a 10% buffered formalin solution for the histological analysis.

Hydroxyproline Measurement

Hydroxyproline liver content was determined as described in Anal. Biochem. 1981; 112: 70-75, with slight modifications.

Liver tissue (0.4 g) was homogenized in 3 ml 6 N HCl and hydrolyzed at 110° C. for 24 h. After centrifugation at 14000 rpm for 10 min, 100 μl of supernatant were neutralized with 50 μl 10N NaOH and 150 μl 1N NaHCO3. After centrifugation at 14000 for 5 min, 100 μl of supernatant were mixed with 200 μl of acetate citrate buffer plus Chloramine T pH 6.0, after incubation for 10 min at RT, 1.3 ml of Ehrlich's reagent was added and the mixture was incubated at 60° C. for 25 min. After cooling, the absorbance was measured at 558 nm (Jasco V-530).

Histopathology and Morphometry

For each case, the median and the left lobes of the liver were subjected to standard trimming and embedding procedures; slides were stained with Hematoxylin and Eosin (H&E) (Laboratory Methods in Histotechnology; Armed Forces Institute of Pathology Published by the American Registry of Pathology Washington, D.C. 1992). Special techniques were also applied in order to allow proper connective tissue detection (Masson's Trichrome stain—Laboratory Methods in Histotechnology; Armed Forces Institute of Pathology Published by the American Registry of Pathology Washington, D.C. 1992) as well as specific collagen detection and hepatic fibrosis evaluation (Syrius red staining method as described in The Journal of Histochemistry and Citochemistry 1985; vol 33, No. 8, pp. 737-743).

Liver fibrosis was qualitatively scored on Syrius red stained slides as follows:

0=Absence of liver fibrosis, but normal staining pattern only;

1=Mild degree, corresponding to centrilobular/periportal fibrosis, with predominantly uncomplete septal expansion—mild focal interstitial fibrosis;

2=Moderate degree, corresponding to diffuse bridging fibrosis, with focal nodular appearance—multifocal interstitial fibrosis;

3=Severe degree, corresponding to confluent fibrosis,

with diffuse nodular appearance—severe diffuse interstitial fibrosis.

The histopathologic findings were detected at the “ECLIPSE E800M” light microscope (Nikon Instruments S.p.A.), equipped with a 3CCD color video-camera JVC “KY F55BE”, connected to a personal computer.

Relevant pictures were stored by the use of a suitable analytical software for image processing and recording (ARKON, A&P Software, Genos—Ark.).

A quantitative analysis of the degree of fibrosis, following the histological qualitative analysis, was performed by digital imaging conversion of the histological pictures by a previously described method (J. Gastroenterol. 2004, Oct. 1; 10(19)2894-2897).

Briefly, after staining with Sirius Red, liver collagen content was measured by histomorphometric method. A series of pictures were made on the whole area of the section (Leica DM-IRE2). Each picture was analyzed by computer system ImageJ version 1.39 for digital image analysis. Detection thresholds were set for the red color of stained collagen. The fibrotic area with positive staining were automatically selected, outlined and evaluated. Relative content of collagen was calculated as a percentage of positive staining pixels on the total number of pixels of the picture.

Results

The results obtained are reported in the following Tables 3-6.

Table 3 shows the body and organ weights, expressed as an index of body weight, in the three groups.

TABLE 3

| Rat | Ctr. Methocel 0.5% | | Ctr. LF Methocel 0.5% | | RLF 1 mg/kg os | |
|---|---|---|---|---|---|---|
| N° | BW g. | Spleen/bw (%) | BW g. | Spleen/bw (%) | BW g. | Spleen/bw (%) |
| 1 | 472 | 0.168 | 360 | 0.295 | 344 | 0.262 |
| 2 | 472 | 0.180 | 395 | 0.269 | 333 | 0.247 |
| 3 | 445 | 0.175 | 327 | 0.281 | 381 | 0.302 |
| 4 | 460 | 0.183 | 329 | 0.495 | 345 | 0.348 |
| 5 | 460 | 0.185 | 344 | 0.351 | 344 | 0.288 |
| 6 | 458 | 0.180 | 353 | 0.403 | 347 | 0.251 |
| 7 | 425 | 0.189 | 338 | 0.389 | 364 | 0.316 |
| 8 | dead | — | dead | — | 366 | 0.261 |
| Mean | 456 | 0.180 | 349.4 | 0.355 | 353 | 0.284 |
| sem | 5.8 | 0.0024 | 8.3 | 0.029 | 5.6 | 0.013 |
| p | | | <0.01 Vs. | <0.01 Vs. | <0.01 vs. | <0.05 vs |
| | | | Ctr. | Ctr. | Ctr. | Ctr LF |

The results reported in Table 3 show that the compound of the invention reduced in statistically significant manner by 20% the spleen weight in rats with CCl₄-induced liver fibrosis.

The weight of the liver and transaminases were not modified after the treatment of the compound of the invention.

Table 4 shows the plasma albuminemia in the three groups.

TABLE 4

| Rat | Ctr. Methocel 0.5% | Ctr. LF Methocel 0.5% | RLF 1 mg/kg os |
|---|---|---|---|
| N° | Albumin g/dl | Albumin g/dl | Albumin g/dl |
| 1 | 3.50 | 2.51 | 2.95 |
| 2 | 3.57 | 2.93 | 3.19 |
| 3 | 3.46 | 3.28 | 3.07 |
| 4 | 3.52 | 3.09 | 3.82 |
| 5 | 3.63 | 2.93 | 4.04 |
| 6 | 3.40 | 3.39 | 3.65 |
| 7 | 3.62 | 3.02 | 3.36 |
| 8 | dead | dead | 4.15 |
| Mean | 3.53 | 3.02 | 3.53 |
| sem | 0.032 | 0.107 | 0.16 |
| p | | <0.05 Vs | <0.05 Vs |
| | | Ctr | Ctr LF |

The results reported in Table 4 show that the compound of the invention increased in statistically significant manner by 16.8% the plasma levels of albumin in rats with CCl₄-induced liver fibrosis.

Table 5 shows the liver hydroxyproline content in the three groups.

TABLE 5

| | Ctr. Methocel 0.5% | Ctr. LF Methocel 0.5% | RLF 1 mg/kg os |
|---|---|---|---|
| Rat | hydroxyproline | hydroxyproline | hydroxyproline |
| N° | μg/g prot | μg/g prot | μg/g prot |
| 1 | 391.8 | 1353.3 | 799.8 |
| 2 | 574.0 | 2254.6 | 2762.3 |
| 3 | 538.5 | 1568.7 | 750.0 |
| 4 | 427.2 | 1564.2 | 1460.7 |
| 5 | 512.7 | 1195.4 | 886.5 |
| 6 | 467.6 | 2393.4 | 791.6 |
| 7 | 656.3 | 845.6 | 1217.4 |
| 8 | dead | dead | 811.0 |
| Mean | 509.72 | 1596.45 | 1184.9 |
| sem | 34.13 | 210.09 | 242.2 |

The results reported in Table 5 show that the compound of the invention reduced in a significant manner (25.8%) the liver content of hydroxyproline, which is an index of collagen deposition, thus fibrosis, in rats with CCl₄-induced liver fibrosis.

Histopathology and Morphometry

The main pathology in CCl₄ treated animals (Ctr.LF group) was represented by remarkable hepatocellular damage (ballooning degeneration and apoptosis, intracytoplasmic inclusion bodies, fatty change), chronic inflammatory reaction, extracellular matrix deposition and regenerative changes (proliferation and hyperplasia of parenchymal and non-parenchymal cells).

A minor extension of tissue damage was shown from rats treated with the Rostafuroxin.

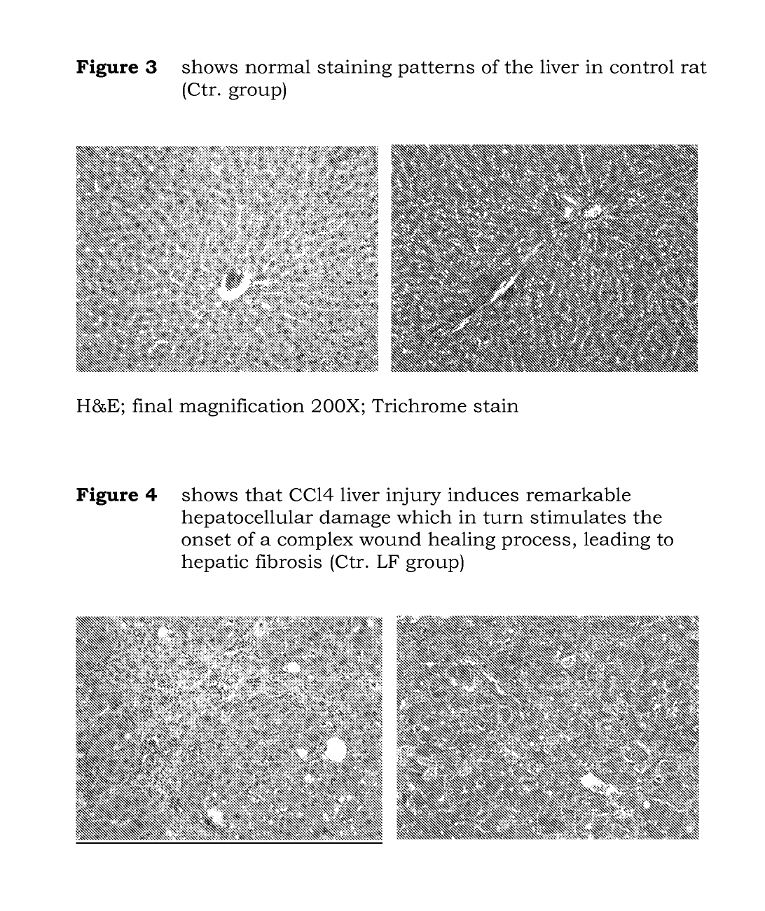

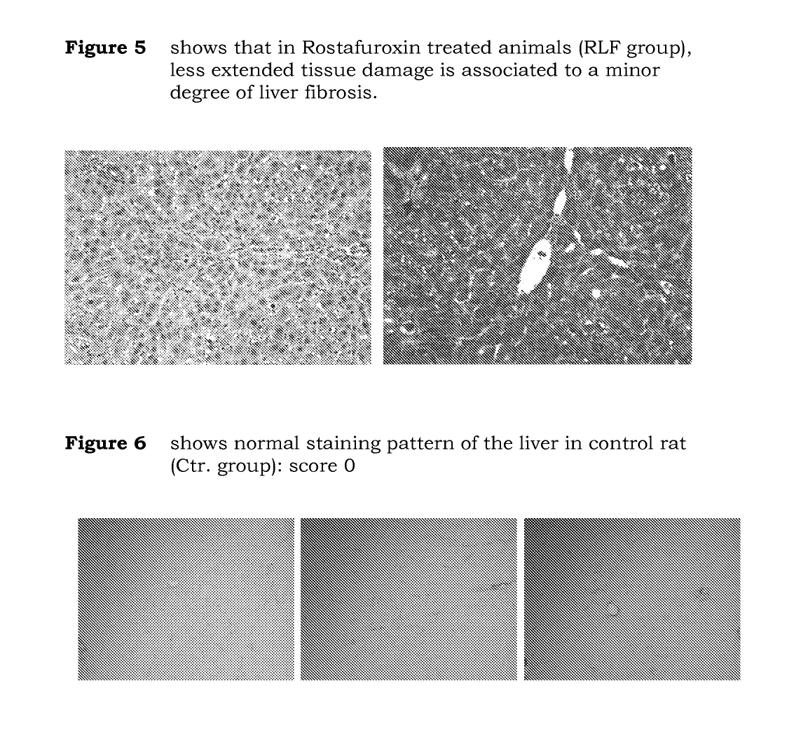

Images of the results obtained are reported in FIGS. 3-5.

Table 6 shows the qualitative evaluation of liver fibrosis, on the basis of the adopted scoring system on Syrius red stained slides

TABLE 6

| | | Ctr. Methocel 0.5% | | Ctr. LF Methocel 0.5% | | RLF 1 mg/kg OS | |
|---|---|---|---|---|---|---|---|
| Liver fibrosis | Score | No. | % | No. | % | No. | % |
| Absence | 0 | 7/7 | 100 | 0/7 | 0 | 0/8 | 0 |
| Mild | 1 | 0/7 | 0 | 0/7 | 0 | 6/8 | 75 |
| Moderate | 2 | 0/7 | 0 | 6/7 | 85.7 | 2/8 | 25 |
| Severe | 3 | 0/7 | 0 | 1/7 | 14.3 | 0/8 | 0 |

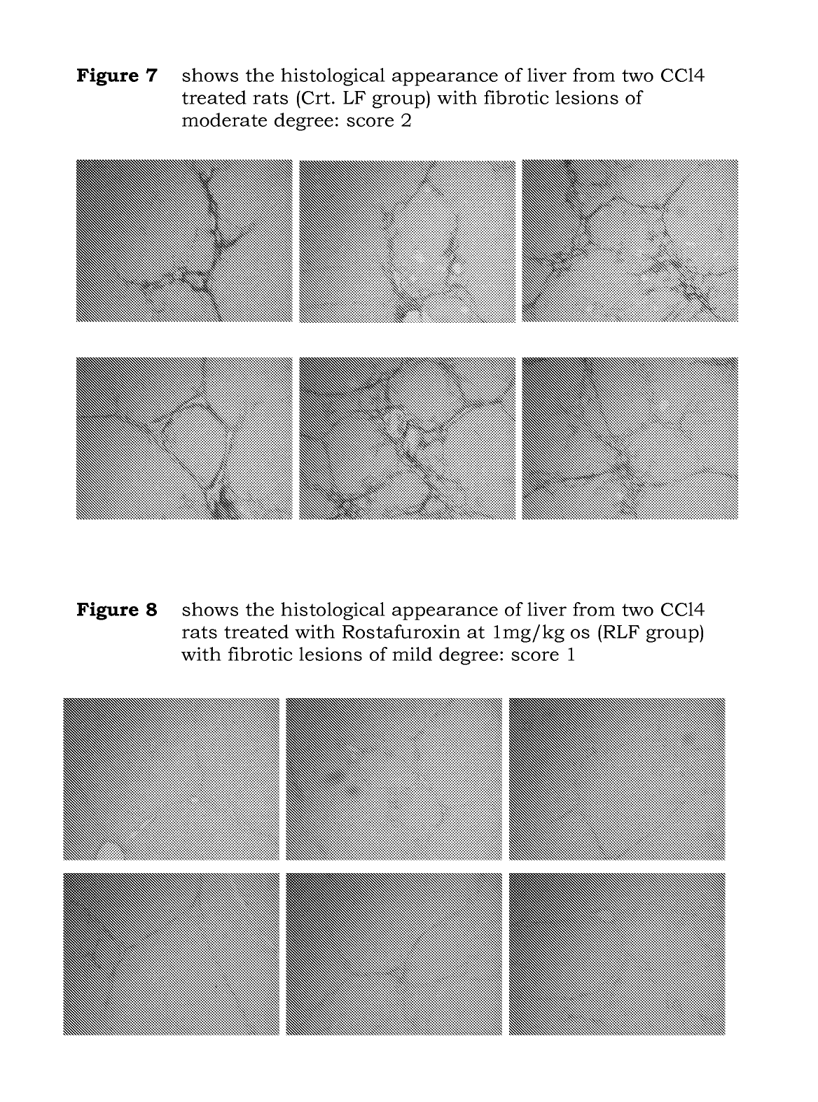

The results reported in Table 6 and FIGS. 6-8 indicate that all animals treated with CCl₄ alone (Ctr. LF group) showed high score values of liver fibrosis, ranging from moderate (85.7% of cases) to severe in degree (one case, corresponding to 14.3%).

Conversely, mild degree of liver fibrosis was detected only in CCl4+Rostafuroxin, the compound of the invention, treated animals (RLF group), and it was observed in the large majority of cases (75%). In the same group, a moderate degree of liver fibrosis was observed in two animals only (25%, compared to the 85.7% of CCl₄ group). Finally, no rats treated with CCl₄+Rostafuroxin showed severe liver fibrosis.

Table 7 shows the quantitative analysis of the degree of liver fibrosis as observed by the analysis in the three groups.

TABLE 7

| Rat | % FIBROTIC AREA | | |
|---|---|---|---|
| n° | Ctr. Methocel 0.5% | Ctr. LF Methocel 0.5% | RLF 1 mg/kg os |
| 1 | 1.43 | 11.55 | 6.63 |
| 2 | 0.57 | 10.40 | 6.89 |
| 3 | 1.74 | 8.42 | 5.08 |
| 4 | 0.61 | 17.18 | 11.80 |
| 5 | 0.28 | 10.26 | 2.01 |
| 6 | 0.52 | 6.97 | 4.64 |
| 7 | 1.47 | 8.57 | 5.50 |
| 8 | | | 1.44 |
| Mean | 0.95 | 10.48 | 5.50 |
| sem | 0.22 | 1.26 | 1.14 |
| p | | <0.01 Vs | <0.05 Vs |
| | | Ctr | Ctr LF |

The results reported in Table 7 show that the compound of the invention reduced in a statistically significant manner by 47.5% the liver fibrotic area in rats with CCl4-induced liver fibrosis.

1. A method for the prevention or treatment of restenosis, said method comprising administering to a mammal in need thereof a compound of formula (I) 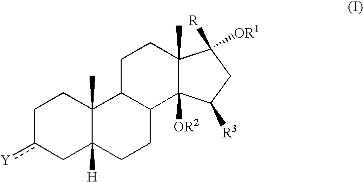
wherein:
the symbol [symbol] represents a single or a double bond;
Y is oxygen or guanidinoimino when [symbol] in position 3 is a double bond;
Y is hydroxy, OR⁴ or SR⁴, when [symbol] in position 3 is a single bond and can have an alpha or beta configuration;
R is an unsubstituted or substituted 3-furyl or 4-pyridazinyl group;
R¹ is hydrogen; methyl; ethyl or n-propyl substituted by OH or NR⁵R⁶;
R² is hydrogen or together to R³ is a bond of an oxirane ring;
R³ is hydrogen or together to R² is a bond of an oxirane ring;
R⁴ is hydrogen; methyl; C2-C6 alkyl or C3-C6 alkenyl or
C2-C6 acyl, these alkyl, alkenyl and acyl groups being unsubstituted or substituted by a quaternary ammonium group or one or more OR⁷, NR⁸R⁹, formyl, amidino, guanidinoimino or by NR⁸R⁹ and hydroxy;
R⁵, R⁶ are independently hydrogen; methyl; C2-C6 alkyl unsubstituted or substituted by one NR¹⁰R¹¹, or NR¹⁰R¹¹ and hydroxy, or R⁵ and R⁶ taken together with the nitrogen atom form an unsubstituted or substituted saturated or unsaturated penta- or hexa-monoheterocyclic ring, optionally containing another heteroatom chosen from oxygen or sulfur or nitrogen;
R⁷ is hydrogen, methyl or C2-C4 alkyl, this alkyl being unsubstituted or substituted by one or more NR¹⁰R¹¹ or by NR¹⁰R¹¹ and hydroxy;
R⁸, R⁹ are independently hydrogen; methyl; C2-C6 alkyl or C3-C6 alkenyl, these alkyl and alkenyl groups being unsubstituted or substituted by one or more NR¹⁰R¹¹, or NR¹⁰R¹¹ and hydroxy, or R⁸ and R⁹ taken together with the nitrogen atom form an unsubstituted or substituted saturated or unsaturated penta- or hexa-monoheterocyclic ring, optionally containing another heteroatom chosen from oxygen or sulfur or nitrogen, or R⁸ is hydrogen and R⁹ is amidino; or NR⁸R⁹ represents propargylamino,
R¹⁰, R¹¹ are independently hydrogen, C1-C6 alkyl, or R¹⁰ and R¹¹, taken together with the nitrogen atom form a saturated or unsaturated penta- or hexa-monoheterocyclic ring.

2. A method for the prevention or treatment of diseases due to organ fibrosis comprising administering the compound of formula (I) of claim 1 to a mammal in need thereof.

3. Method according to claim 1, wherein said compound of formula (I) of claim 1 is selected from the group consisting of:
17 beta-(3-Furyl)-5 beta-androstane-3 beta, 14 beta, 17 alpha-triol;
3 beta-(2-Hydroxyethoxy)-17 beta-(3-furyl)-5 beta-androstane-14 beta, 17 alpha-diol;
3 beta-(2-Aminoethoxy)-17 beta-(3-furyl)-5 beta-androstane-14 beta, 17 alpha-diol;
3 beta-(3-Aminopropoxy)-17 beta-(3-furyl)-5 beta-androstane-14 beta, 17 alpha-diol;
3 beta-(2-Methylaminoethoxy)-17 beta-(3-furyl)-5 beta-androstane-14 beta, 17 alpha-diol;
3 beta-(2-(1-Pyrrolidinyl)ethoxy)-17 beta-(3-furyl)-5 beta-androstane-14 beta, 17 alpha-diol;
3 beta-(2-(3-(1-Pyrrolidinyl)propoxy)ethoxy)-17 beta-(3-furyl)-5 beta-androstane-14 beta, 17 alpha-diol;
3 beta-(3-(1-Pyrrolidinyl)propoxy)-17 beta-(3-furyl)-5 beta-androstane-14 beta, 17 alpha-diol;
3 beta-(2-(1-Imidazolyl)ethoxy)-17 beta-(3-furyl)-5 beta-androstane-14 beta, 17 alpha-diol;
3 beta-(2-(2-Imidazolin-2-yl)ethoxy)-17 beta-(3-furyl)-5 beta-androstane-14 beta, 17 alpha-diol;
3 beta-(2-(2-Amidino)ethoxy)-17 beta-(3-furyl)-5 beta-androstane-14 beta, 17 alpha-diol;
3 beta-(2-(2-(1-Pyrrolidinyl)ethoxy)ethoxy)-17 beta-(3-furyl)-5 beta-androstane-14 beta, 17 alpha-diol;
3 beta-(2-Guanidinoethoxy)-17 beta-(3-furyl)5 beta-androstane-14 beta, 17 alpha-diol;
3 beta-(3-Guanidinopropoxy)-17 beta-(3-furyl)-5 beta-androstane-14 beta, 17 alpha-diol;
3 beta-(3-Amino-2-hydroxypropoxy)-17 beta-(3-furyl)-5 beta-androstane-14 beta, 17 alpha-diol;
3 beta-(2,3-Diaminopropoxy)-17 beta-(3-furyl)5 beta-androstane-14 beta, 17 alpha-diol;
17 beta-(3-Furyl)-17 alpha-methoxy-5 beta-androstane-3 beta, 14 beta-diol;
17 beta-(3-Furyl)-17 alpha-(2-(1-pyrrolidinyl)ethoxy)-5 beta-androstane-3 beta, 14 beta-diol;
17 beta-(3-Furyl)-17 alpha-(3-aminopropoxy)-5 beta-androstane-3 beta, 14 beta-diol;
3 beta-(2-(1-Pyrrolidinyl)ethoxy)-17 beta-(3-furyl)-17 alpha-methoxy-5 beta-androstan-14 beta-ol;
3 beta, 17 alpha-Bis(2-(1-pyrrolidinyl)ethoxy)-17 beta-(3-furyl)-5 beta-androstan-14 beta-ol;
3 beta, 17 alpha-Bis(3-aminopropoxy)-17 beta-(3-furyl)-5 beta-androstan-14 beta-ol;
14 beta, 17 alpha-Dihydroxy-17 beta-(3-furyl)-5 beta-androstan-3-one;
3-Guanidinoimino-17 beta-(3-furyl)-5 beta-androstane-14 beta, 17 alpha-diol;
17 beta-(4-Pyridazinyl)-5 beta-androstane-3 beta, 14 beta, 17 alpha-triol;
3 beta-(2-Hydroxyethoxy)-17 beta-(4-pyridazinyl)5 beta-androstane-14 beta, 17 alpha-diol;
3 beta-(3-Aminopropoxy)-17 beta-(4-pyridazinyl)-5 beta-androstane-14 beta, 17 alpha-diol;
3 beta-(2-(1-Pyrrolidinyl)ethoxy)-17 beta-(4-pyridazinyl)-5 beta-androstane-14 beta, 17 alpha-diol;
3 beta-(3-(1-Pyrrolidinyl)propoxy)-17 beta-(4-pyridazinyl)-5 beta-androstane-14 beta, 17 alpha-diol;
17 beta-(4-Pridazinyl)-17 alpha-(3-aminopropoxy)-5 beta-androstane-3 beta, 14 beta-diol;
3 beta-(2-(1-Pyrrolidinyl)ethoxy)-17 beta-(4-pyridazinyl)-17 alpha-methoxy-5 beta-androstan-14 beta-ol;
3 beta-(2-(1-Pyrrolidinyl)ethoxy)-17 beta-(4-pyridazinyl)-17 alpha-(3-amino-propoxy)-5 beta-androstan-14 beta-ol;
14 beta, 17 alpha-Dihydroxy-17 beta-(4-pyridazinyl)-5 beta-androstan-3-one;
3-Guanidinoimino-17 beta-(4-pyridazinyl)-5 beta-androstane-14 beta, 17 alpha-diol;
14 beta, 15 beta-Epoxy-17 beta-(3-furyl)-5 beta-androstane-3 beta, 17 alpha-diol;
3 beta-(2-Hydroxyethoxy)-14 beta, 15 beta-epoxy-17 beta-(3-furyl)-5 beta-androstan-17 alpha-ol;
3 beta-(3-Aminopropoxy)-14 beta, 15 beta-epoxy-17 beta-(3-furyl)-5 beta-androstan-17 alpha-ol;
3 beta-(2-(1-Pyrrolidinyl)ethoxy)-14 beta, 15 beta-epoxy-17 beta-(3-furyl)-5 beta-androstan-17 alpha-ol;
3 beta-(3-(1-Pyrrolidinyl)propoxy)-14 beta, 15 beta-epoxy-17 beta-(3-furyl)-5 beta-androstan-17 alpha-ol;
3 beta-(2-(1-Pyrrolidinyl)ethoxy)-17 beta-(3-furyl)-17 alpha-methoxy-14 beta, 15 beta-epoxy-5 beta-androstane;
17 alpha-Hydroxy-17 beta-(3-furyl)-14 beta, 15 beta-epoxy-5 beta-androstan-3-one;
3-Guanidinoimino-17 beta-(3-furyl)-14 beta, 15 beta-epoxy-5 beta-androstan-17 alpha-ol;
14 beta, 15 beta-Epoxy-17 beta-(4-pyridazinyl)-5 beta-androstane-3 beta, 17 alpha-diol;
3 alpha derivatives of the 3 beta derivatives and their corresponding 3 alpha and 3 beta thioderivatives where Y═S.

4. A method for preparing a medicament for the prevention and/or treatment of obstructive vascular lesions following vascular surgery or diseases due to organ fibrosis, said method comprising administering the compound of formula (I) of claim 1 to a mammal in need thereof.

5. Method according to claim 4, wherein said compound is 17 beta-(3-Furyl)-5 beta-androstane-3 beta, 14 beta, 17 alpha-triol.

6. Method according to claim 4, wherein vascular surgery is selected from the group consisting of angioplasty, percutaneous transluminal coronary angioplasty, by-pass grafting, endartherectomy and stent implantation.

7. Method according to claim 4, wherein the obstructive vascular lesion is restenosis after angioplastic or after endoartherectomy.

8. Method according to claim 4, wherein the disease due to organ fibrosis is selected from the group consisting of kidney fibrosis; heart fibrosis; pancreas fibrosis; lung fibrosis; vascular vessel fibrosis; skin fibrosis; bone marrow fibrosis liver fibrosis; pachyderma, keloid and systemic sclerosis.

9. Method according to claim 8, wherein liver fibrosis is due to a virus disease, alcoholic hepatitis, non-alcoholic steatohepatitis, cirrhosis or liver cancer.

10. Method according to claim 4, wherein the compound is administered in a dose of from 0.05 mg to 20 mg per day.

11. Method according to claim 4, wherein the compound is administered in a dose of from 0.5 mg to 15 mg.

12. Method according to claim 4, wherein the compound is administered in a dose of from 5 mg to 10 mg.

13. Method according to claims 4, wherein the compound is administered in a single dose schedule.

14. Method according to claims 4, wherein the compound is administered in a multiple dose schedule.

15. Method according to claim 4, wherein the composition is for oral, intravenous, intramuscular, intra-arterial, intramedullary, intrathecal, intraventricular, transdermal, transcutaneous, subcutaneous, intraperitoneal, intranasal, enteral, topical, sublingual or rectal administration.

16. Method according to claim 4, wherein the composition is administered locally on the diseased tissue after surgical operation.

17. Method according to claim 4, wherein the composition is coated on the stent; incorporated into a controlled-release matrix; or incorporated into liposomes.
